(19) United States
(12) Patent Application Publication
(10) Pub. No.: US 2004/0147532 A1
(43) Pub. Date: Jul. 29, 2004

(54) Liquid conjugates of solid pharmaceuticals

(75) Inventors: Shalaby W. Shalaby, Anderson, SC (US); Jaymin C. Shah, Waterford, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/693,307

(22) Filed: Oct. 24, 2003

Related U.S. Application Data
(60) Provisional application No. 60/422,833, filed on Oct. 31, 2002.

(51) Int. Cl. A61K 31/519; A61K 9/14

(52) U.S. Cl. 514/259.41; 424/486

(57) ABSTRACT

A liquid phase conjugate formed of a bioactive agent such as a drug compound, e.g. ziprasidone, and a liquid polymer of requisite functionality is disclosed.

### FIELD OF THE INVENTION

This application claims priority under 35 USC 119(e) of U.S. Provisional 601422,833, filed Oct. 31, 2002.

The invention relates to a conjugate comprised of a pharmaceutical compound and an absorbable polymer. The conjugate of the invention is liquid so as to facilitate its formulation into various dosage forms, such as solid and liquid dosage forms, including injectable depot formulations.

### BACKGROUND OF THE INVENTION

Most bioactive agents, which include pharmaceutical compounds, are produced as amorphous or as crystalline solids having variable thermal properties and solubilities in aqueous or lipophilic vehicles. Based on these properties, among other things, most bioactive agents are formulated into solid or liquid dosage forms using liquid or solid vehicles commensurate with their solubilities, as well as with other processing additives, and/or excipients to provide for administration to a patient by oral, parenteral or other routes.

Solubility of a bioactive agent can be increased in a liquid formulation using one of the following: 1. Cosolvents, or 2. Surface active agents and/or complexing agents such as macrocyclic cage compounds. However, it can be difficult to control the release of the bioactive agent from such a solution upon administration to a patient by oral, parenteral or other route,

Various means to provide sustained release of poorly soluble bioactive agents in a liquid formulation include some of the following examples: 1. Dissolving or dispersing lipophilic drugs in oils, 2. Dispersing solid drugs in absorbable liquid polymers, or 3. Dispersing or dissolving solid drugs in absorbable gel-forming liquids. See e.g. U.S. Pat. Nos. 5,653,992; 5,714,159; 6,413,539; and 5,612,652. Meanwhile, to prolong the in vivo half life of bioactive peptides and proteins, and to control their release profile and bioavailability, water-insoluble ionic conjugates with absorbable polymeric chains have been developed, which can be formulated as injectable, aqueous dispersions. See e.g. U.S. Pat. Nos. 5,672,659; 5,665,702; 5,821,221; 5,863,985; 5,916,883; 6,204,256; and 6,221,958.

Despite these efforts, the following formulation issues can still be problematic: 1. Uniformity of a solid active agent in a dispersion, 2. Limitations associated with low concentration of an active agent in a liquid vehicle due to poor solubility, and 3. Concerns associated with the fate of a liquid vehicle that represents a major component of a parenteral formulation. These concerns are particularly pressing where the bioactive agent is in a solid form. In this regard, it is particularly desirable to be able to constitute said solid bioactive agent into a liquid formulation with higher solubility but that provides slow release; a liquid formulation of such a kind would facilitate syringeability and enable incorporation of said agent into parenteral and like dosage forms.

There is thus a need for a dosage formulation that addresses the foregoing problems, including, without limitation, in circumstances wherein the bioactive agent is a solid pharmaceutical compound that is insoluble or poorly soluble in water.

### SUMMARY OF THE INVENTION

The present invention is directed to the foregoing need. In one aspect, the invention pertains to a liquid conjugate comprising a bioactive agent and an absorbable liquid polymer, said bioactive agent and said absorbable liquid polymer being at least partly ionically linked together to form said liquid conjugate.

### DETAILED DESCRIPTION OF THE INVENTION

The invention relates to conjugates formed at least by the following conjugate components: a bioactive agent; and a liquid polymer. The bioactive agent and absorbable liquid polymer are linked together, at least in part, ionically. In one embodiment, the conjugates of the invention have a select percentage of ionic linkage and lead to improved aqueous solubility of the active agent and improved dispersiveness and delivery when constituted into a pharmaceutical formulation. In a general practice, the solid bioactive agent has either basic or acidic aspects or moieties; the liquid polymer having the opposite character. Thus without limitation, when the bioactive agent is basic, e.g. has amine groups, the liquid polymer is acidic, e.g. has carboxyl groups; when the bioactive agent is acidic, the liquid polymer is basic. As appreciated by the artisan, these groups must be sufficiently accessible to provide the select ionic linkage envisioned by the invention.

In one practice, the liquid conjugates of the invention can be employed to increase the solubility of a drug compound, even drug compounds that are already soluble.

In a preferred practice, the liquid conjugates of the invention are used in formulating dosage forms for water insoluble or poorly soluble drugs.

In any instance, dosage forms in which the liquid conjugates of the invention have application include, without limitation, oral formulations, e.g. suspensions, tablets, capsules and the like; and injectable formulations, e.g. intramuscular injection and the like. Other dosage forms in which the invention can be used include, without limitation, immediate release and controlled release formulations, such as depot formulations including, without limitation, intramuscularly injectable depot formulation of, for example, ziprasidone. Such formulations can be used to treat mammals, including humans, in need of treatment for illnesses, for example schizophrenia and other psychotic disorders.

Bioactive Agent:

The term “bioactive agent” is readily understood by the artisan. Without limitation, the term includes pharmaceutical compounds (organic molecules) (also referred to herein as “drug(s)” or“drug compound(s)” including variations of same), and pharmaceutical peptides or proteins—all of which are used herein interchangeably with the term “bioactive agent.” In a preferred practice the bioactive agent is in solid form. Bioactive agents contemplated for use in the invention can be natural or synthetic, acidic, or basic. Basic bioactive agents are preferred, including e.g. those that are amine-containing, i.e. those containing one or more amine groups. Other basic bioactive agents contemplated for use with the invention are basic drugs that are simple organic compounds having a molecular weight of more than 150 Da. The drug can also be a peptide comprising at least two amino-acid sequences, or it can be a protein.

Without limitation, the bioactive agent used in the present invention is, in one embodiment, an aryl-heterocyclic compound, particularly chosen from those having psychotropic effects, such as the chlorooxyindole class of such heterocyclics. Representative aryl-heterocyclic compounds for purposes of this invention are those described in U.S. Pat. No. 4,831,031, incorporated herein by reference. In a particular practice the drug in question is ziprasidone, i.e. 5-[2-[4-(1,2-benzisothiazol-3-yl)-1-piperazinyl]ethyl]-6-chloro-1,3-dihydro-2H-indol-2-one. The ziprasidone can be in a pharmaceutically acceptable salt form in the practice of the invention; preferably it is in its free base form, which is known to be insoluble or poorly soluble in water.

Bioactive agents that can be used in the present invention may also be soluble in traditional organic solvents such as ketones (e.g. acetone), nitriles (e.g. acetonitrile), and hydrocarbons (e.g. chloroform).

Liquid Polymers:

The liquid polymers of the invention are functionalized, e.g. are those bearing moieties that provide suitable ionic attraction with the drugs aforesaid to generate the ionic bonding whereby the conjugates of the invention form. Such moieties include those that render the polymer acidic, e.g. carboxyl groups; or basic, e.g. amine groups. Without limitation, such polymers include carboxyl-bearing polyesters, copolyesters, polyalkylene carbonates and copolyester-carbonates; and amine-bearing polyesters, copolyesters, polyalkylene carbonates, polyether carbonates, polyethers, and copolyester-carbonates. It is preferred if the acidic or basic groups of the functional polymer are sufficiently accessible for purposes of forming the select ionic linkage of the inventive conjugate, e.g. in the case of ziprasidone, that the acidic functional polymer has reasonably accessible carboxylic groups, for example. The polymers of the invention are absorbable, i.e. they are pharmaceutically acceptable and are biodegradable. The polymers of the invention are also in the liquid state as before stated. Without limitation and as appreciated by the artisan, such polymers include those that are more hydrophilic, and/or have shorter chain lengths, or have structure similar to those of pluronics as compared to solid polymers.

Ionic Conjugation:

Representatively, the liquid conjugate of the invention may be made as follows: the solid bioactive agent is contacted with one or more liquid polymers described above under conditions effective to cause sufficient proton transfer whereby ionic conjugation between the basic aspects or moieties of said drug (or said polymer as the case may be) and said acidic aspects or moieties of said polymer (or the drug as the case may be) occurs. In a preferred practice, the solid bioactive agent is combined, e.g. admixed, with a liquid absorbable polymer such that at least about 50% of the interaction between the two (i.e. between the acidic and basic moieties of the two) is ionic bonding; more preferably about 80% or more of said interaction is ionic bonding. In one aspect, the invention pertains to a liquid conjugate comprising a bioactive agent and an absorbable liquid polymer as conjugate components wherein at least 50% of the conjugate components are bonded tonically; in another embodiment, said liquid conjugate in this regard is a composition. The drug loadings in any given liquid conjugate of the invention can be varied by percentages as appreciated by the artisan.

As used herein the term “conjugate component(s)” refers to (i) the solid bioactive agent and (ii) the absorable liquid polymer.

As used herein the term “mgA/ml” relates to the weight (in mg) of the pharmaceutical compound, calculated in its free form, per ml of composition under consideration. (For ziprasidone as free base, the molecular weight=412.9).

Other aspects of the liquid conjugate of the invention are, without limitation, as follows: One aspect of this invention deals with an absorbable carboxyl-bearing liquid polymer and amine-containing drug. Another aspect of the invention deals with an absorbable carboxyl-bearing liquid polymer and a bioactive agent that contains one or more amine group. In another aspect of this invention, the polymer is a copolyester with more than one carboxyl group. In another aspect of the invention, the polymer comprises polyether and polyester segments that carry more than one carboxyl group per chain. In another aspect of this invention, the segmented polyether-ester chain of the polymeric component carries multiple carboxyl groups. Another aspect of this invention deals with a basic drug that is a simple organic compound having a molecular weight of more than 150 Da. The drug can also be a peptide comprising at least two amino-acid sequences or a protein. Another aspect of this invention deals with a carboxyl-bearing drug that is ionically conjugated to an amine-bearing polymer. The amine-bearing polymer can have a triaxial polyester, polycarbonate, or polyester-carbonate chain with a central tertiary amine group. Another aspect of this invention deals with an absorbable polymeric liquid cation-exchanger comprising sulfonic- or phosphonic-acid as side or terminal groups on their chains. Another aspect of this invention deals with a carboxylated homopolymeric or copolymeric polyalkylene oxide having one or more carboxyl group per chain. Another aspect of this invention deals with ionic conjugates where the mass of the bioactive component constitutes at least 1 percent of the conjugate. Another aspect of this invention deals with a liquid, mostly-ionic conjugate of an absorbable copolyester and a bioactive compound where the mass of the latter constitutes at least 1 percent of the total mass. In a specific aspect of this invention, the liquid conjugate is made by the interaction of a basic bioactive substance, e.g. dipyridamole or ziprasidone, and one of the following liquid absorbable polymers: (1) a carboxyl-bearing polyether, such as polyethylene glycol or a copolymer of polyethylene glycol and polypropylene glycol, grafted with one or more of these monomers: E-caprolactone, trimethylene carbonate, glycolide, lactide, p-dioxanone, 1,5-dioxepan-2-one; or, preferably, monomers containing C-succinic acid side groups; or (2) a copolyester made by the polymerization of one or more cyclic monomer such as trimethylene carbonate, e-caprolactone, 1,5 dioxapan-2-one, lactide, or p-dioxanone, using an initiator such as glycolic, malic, tartaric, citric, lactic, ascorbic and/or gluconic acids. Another aspect of this invention deals with a conjugate of a basic drug and a carboxylic, phosphonic, or sulfonic acid-bearing copolypeptide wherein a fraction of peptide sequences is N-alkylated.

Pharmaceutical Formulations:

Without limitation, the liquid conjugate of the invention is useful in a pharmaceutical formulation. Contemplated formulations include without limitation immediate release and controlled release formulations, especially a controlled release formulation, such as a depot formulation, including without limitation injectable depot formulations, e.g. intramuscularly injectable depot formulations of ziprasidone. The formulations may be for administration by oral, injection or topical routes. The formulations herein can be used to treat mammals, including humans, in need of treatment for, including but not limited to, schizophrenia or another psychotic disorder.

Dosage forms other than injectable are also contemplated herein. Without limitation, the ionic conjugates of the invention can be used to make other dosage forms such as, by way of example only, oral suspensions, topical application forms, tablets, capsules and the like, including, without limitation, immediate release; and controlled release forms, such as injectable depot formulations for intramuscular administration. Controlled release includes, without limitation, the effect of modulating the release of the drug after administration to a mammal.

In a preferred embodiment, the drug is ziprasidone and the liquid polymer is a pluronic polymer, preferably a carboxyl-bearing block/segmented copolymer comprising a polyalkylene carbonate and a polyalkylene oxide segment/block.

Without limitation, the present invention can provide an injectable depot formulation for delivery of e.g. an aryl heterocyclic active agent, such as ziprasidone, at concentrations effective for treatment of illnesses such as schizophrenia over a sustained period of time, i.e. for a period of time beyond that which is obtained by immediate release injection systems. By way of example only, the present invention can provide efficacious plasma levels of active agent, e.g. ziprasidone, for at least 8 hours using typical injection volumes, e.g. about 0.1 ml to about 3 ml., about 1 ml to about 2 ml being usual. Preferably, the sustained period provided by the invention is at least 24 hours; more preferably up to about 1 week; still more preferably from about 1 week to about 2 weeks or more including up to about 8 weeks using the injection volumes aforesaid. For example, in the case of ziprasidone, the practice of the invention can deliver at least about 1 to about 700 mgA, preferably to about 350 mgA, in an injection volume of about 1-2 ml for about 1 to about 2 weeks or more, including up to about 8 weeks. More preferably, about 10 to about 140 mgA for up to about 2 weeks is deliverable.

The invention will for convenience now be further described using ziprasidone as the bioactive agent in the context of the following examples. It will be understood that the examples are illustrative and do not in any way constrain the scope of the invention. Modifications to same as appreciated by the artisan are also contemplated herein.

### EXAMPLE 1

### Preparation of a Liquid Carboxyl-bearing Copolyester

A mixture of dl-lactide (0.4 mole, 57.6 g), polycarbonate glycolide (0.1 mole, 11.6 g), dl-malic acid (0.065 mole, 8.71 g), and stannous octanoate (0.55 ml of 0.2 M solution in toluene) were charged into a pre-dried glass apparatus that was equipped for mechanical stirring. The polymerization was conducted at 160° C. for 3 hours under dry nitrogen atmosphere. At the conclusion of the polymerization period, the product was analyzed by gel-permeation chromatography (GPC) to assure maximum conversion. This was followed by evaporation of trace amounts of residual monomers by heating at 110° C. under reduced pressure. The identity of the purified liquid polymer was confirmed by IR and NMR. The GPC data (in dichloromethane) indicated an Mn=1360 Da and Mw=1930 Da. The resultant liquid polymer was designated A.

### EXAMPLE 2

### Preparation of Ziprasidone Ionic Conjugate With Liquid Polymer A

Free ziprasidone base (1.2 mmole, 501.6 mg) was dissolved in hexafluoroisopropyl alcohol (HFIP, 6 ml). To this solution, the liquid polymer A (1.2 mmole, based on Mn by GPC, 1639 mg) and HFIP (2 ml) were added. After agitiation to obtain a uniform solution, HFIP was evaporated under reduced pressure. The resulting liquid conjugate was analyzed for thermal transition by differential scanning calorimetry (DSC) to verify the absence of the ziprasidone melting endotherm. The identity of the liquid conjugate was confirmed using IR and NMR.

### EXAMPLE 3

### Preparation and Characterization of Hydroxy Acid-Initiated Copolymers (B-Type Polymers)

Copolymers made from cyclic monomers and malic or citric acid as the initiators were prepared and characterized for use in producing liquid conjugates as outlined in Table I. All polymers were liquids at room temperature. The polymers were characterized for carboxyl content (titration), molecular weight (GPC), and complex viscosity (rheome try). The respective data in Table I also show that the equivalent weight, Mₙ and viscosity can be controlled readily by the comonomer composition and amount of malic or citric acid used in the preparation of the polymers.

### Preparation of Low Viscosity End-Grafted PEG Copolyester (C)

This polymer was prepared for use as a diluent for high viscosity conjugates made from B-type copolymers. The P2 polymer was prepared by end-grafting a mixture of trimethylene carbonate (TMC) and glycolide (G) onto PEG-400 as per the following ratio: PEG/(TMC:G)=80/20 (90:10).

TABLE 1
Composition and Properties of Hydroxy Acid-initiated Copolymers

| | | | | Complex | | | |
|---|---|---|---|---|---|---|---|
| | | | | Viscos- | | | |
| | Molar Ratio | | Equivalent | ity @ | GPC Data | | |
| Poly- | Initi- | Cyclic | Weight | 37° C. | M_{n,} | M_{w,} | |
| mer^{a} | ator^{b} | Monomers^{c} | g/Eq.* | Pa · S | kDa | kDa | PDI |
| D | 5 | TMC/G | 1900 | 850 | 5.0 | 9.0 | 1.85 |
| | | 85/10 | | | | | |
| E | 10 | 85/5 | 1500 | 537 | 3.4 | 6.8 | 2.01 |
| F | 5 | 90/5 | 2100 | 270 | 5.2 | 10.7 | 2.06 |
| G | 20 | 78/2 | 900 | 131 | 2.2 | 3.9 | 1.77 |
| H | 23 | 75/2 | 700 | 127 | 2.1 | 3.4 | 1.68 |
| I | 30 | 68/2 | 565 | 164 | 1.8 | 2.7 | 1.51 |
| J | 35 | 63/2 | 383 | 248 | 1.6 | 2.3 | 1.47 |
| K | 11.5 | LL/G | 366 | — | 1.4 | 2.0 | 1.45 |
| | | 70.5/18 | | | | | |
| L^{b} | 9 | 73/18 | | — | 1.7 | 2.4 | 1.45 |
| M^{b} | 11.5 | 70.5/18 | | — | 1.4 | 1.9 | 1.37 |
| N^{b} | 30 | TMC/G/CL | 342 | — | 1.6 | 2.3 | 1.43 |
| | | 50/2/18 | | | | | |

^{a}All polymers were purified by distilling residual monomer under reduced pressure.
^{b}The initiator is malic acid with the exception of L and M where citric acid is used.
^{c}TMC = trimethylene carbonate; G = glycolide; CL = caprolactone; LL = I-lactide.
*Mass of chain per carboxylic group.

### EXAMPLE 4

### Preparation and Characterization of Conjugates of B-Type Polymers

Conjugates of B-type polymers with 10 to 35% ziprasidone were prepared and characterized by IR, DSC, and NMR. Relevant composition data of the conjugates and their physical properties are summarized in Table II. All conjugates were prepared using solutions of the polymer and drug in HFIP. Evaporation of HFIP under reduced pressure was pursued to obtain the pure conjugate. With the exception of TWELVE, traces of HFIP were removed by co-distillation with added chloroform. The characterization data in Table II and other related data show that (1) in almost all cases, with the exception of EIGHT, the drug is incorporated in the conjugate and no free drug could be detected (no discernable Tₘ of the free drug at about 229° C.); (2) the conjugates exhibit endothermic changes during heating in the DSC apparatus which can be related to dissociation and/or decomposition of their constituents; (3) NMR and IR can be used only semi-quantitatively to determine the composition.

TABLE II
Composition and Properties of Conjugates of B-type Polymers
with Ziprasidone

| | | | Key Conjugate Properties | | | | |
|---|---|---|---|---|---|---|---|
| | | | | | DSC Data | | |
| | | | | | Distinct & | | |
| | | | | | Complex^{d} | | |
| | B-type | % | | Diluent | Endotherms | | zip |
| Conjugate | Polymer^{a} | ziprasidone | Consistency* | Added | ° C. | J/g | Tₘ^{b} |
| ONE | 2-2 | 25 | C | No | 127 +^{d} | 21.9 | None |
| TWO | 2-2 | 30 | | No | 123 +^{d} | 32.3 | None |
| THREE | 5-2 | 35 | | No | 124 +^{d} | 32.5 | None |
| FOUR | 6-2 | 35 | | No | 129 +^{d} | 21.8 | None |
| FIVE | 1-A-1a | 10 | B | No | 142 +^{d} | 9.24 | None |
| SIX | 1-G-1 | 10 | B | No | 147 +^{d} | 6.0 | None |
| SEVEN | 1-G-1 | 20 | B | No | 127 +^{d} | 5.25 | None |
| EIGHT^{c} | 1-F-1 | 15 | C | Yes (30%) | 154 +^{d} | 4.5 | Yes |
| NINE | 1-F-1 | 15 | C | Yes (30%) | 174 +^{d} | 3.4 | None |
| TEN | 1-F-1 | 15 | C | Yes (15%) | 160 +^{d} | 9.6 | None |
| ELEVEN^{e} | 9-1 | 15 | C | No | 171 +^{d} | 8.43 | None |
| TWELVE | 9-1 | 15 | C | No | — | — | — |

*A = Gummy; B = Viscous Liquid; C = Fluid Liquid.
^{a}For composition and analytical data, see Table I.
^{b}zip Tₘ at about 229° C.
^{c}Heat-dried under reduced pressure.
^{d}Complex endotherm and respective J/g, could not be determined with certainty.
^{e}TWELVE and ELEVEN, both were prepared aseptically; TWELVE was isolated prior to chloroform treatment.

### EXAMPLE 5

### Preparation and Characterization of C-succinylated Polyether-esters (O-Type Copolymer)

Polyethylene glycols PEG400 and PEG-600 were end-grafted with mixtures of trimethylene carbonate (TMC) and caprolactone (CL) to produce liquid copolyesters. These were reacted with maleic anhydride under free-radical conditions. The anhydride group of the resulting product was hydrolyzed selectively to produce C-succinylated liquid polymers (O-type). These were made for use in preparing liquid conjugates with ziprasidone. The O-polymers were characterized for composition (NMR, IR), carboxyl content (titration), and molecular weight (GPC). The respective data are outlined in Table III. All copolymers were liquids with varying viscosities at room temperature. The data in Table III show that the (1) molecular weight can be controlled by the type and amount of PEG used; and (2) molecular weight distributions of the PEG-400-based copolymers are higher than those of PEG-600 counterparts.

TABLE III
Composition and Properties of C-succinylated Polyether-esters

| | Composition, Mole % | | | | | | |
|---|---|---|---|---|---|---|---|
| | | | Theoretical | | | | |
| | | Precursor | Number of | Equivalent | | | |
| Polymer | PEG- | Molar Ratio^{b} | Carboxyl | Weight, | GPC Data | | |
| Number^{a} | type | PEG/(TMC:CL) | Groups^{c} | g/Eq. | Mₙ, kDa | M_{w}, kDa | PDI |
| O-1 | 400 | 23/(62/15) | 3 | 1121 | 4.2 | 10.7 | 2.58 |
| O-2 | 400 | 23/(61/15) | 3 | 1246 | 3.8 | 14.9 | 3.89 |
| O-3 | 600 | 17/(66/17) | 3 | 1344 | 2.7 | 4.0 | 1.47 |
| O-4 | 600 | 17/(66/17) | 5 | 604 | 2.5 | 3.5 | 1.40 |
| O-5 | 600 | 17/(66/17) | 4 | 1324 | 3.4 | 5.0 | 1.46 |

^{a}All polymers were purified by distilling residual monomer under reduced pressure.
^{b}PEG - Polyethylene glycol (400 or 600); TMC = trimethylene carbonate; CL = caprolactone.
^{c}Expected number of succinic acid-derived carboxyl groups per chain.

### EXAMPLE 6

### Preparation and Characterization of Liquid Conjugates and Controls Using the O-Type Copolymers and Their Intermediates

The conjugates were prepared under similar conditions to those used in the preparation of B-based systems. Carboxyl-free intermediates or precursors (e.g.,precursors to O-1 and O-2) of O-type copolymer (prior to the maleation process) were used to prepare control systems (Controls I and II), which are expected to be incapable of conjugate formation. Control II was prepared by mixing HFIP solutions of the precursor (O-type precursor) and ziprasidone, while Control I was made by adding the polymeric precursor (O-type precursor) directly to the ziprasidone solution in HFIP. The conjugates and their controls were characterized as described for the B-based system. Critical composition and analytical data are summarized in Table IV. The data in Table IV and other relevant results indicate that (1) the O-polymers are indeed capable of forming liquid conjugates with ziprasidone; (2) a carboxyl-free precursor (O-type precursor) of a typical O polymer is incapable of forming conjugates with ziprasidone and free drug undergoes precipitation from its solution in the presence of the O-type precursor; (3) both PEG-400- and PEG-600-based O-copolymer are suitable for preparing liquid conjugates; and (4) up to 20% ziprasidone can be incorporated into a liquid conjugate without the need for a diluent polymer to reduce the viscosity.

TABLE IV
Composition and Properties of EC-type Polymers with ziprasidone

| | | | Key Conjugate Properties | | | |
|---|---|---|---|---|---|---|
| | | | | DSC Data | | |
| | | | | Distinct & | | |
| | | | | Complex^{d} | | zip, |
| Conjugate | O-type | % | Consis- | Endotherms | | Tₘ, |
| Number | Polymer^{a} | zip | tency* | ° C. | J/g | ° C.^{b} |
| THIRTEEN | O-1 | 20 | B | 134 +^{d} | 22.1 | None |
| FOURTEEN | O-2 | 20 | B | 138 +^{d} | 20.9 | None |
| Control I^{c} | O-precursor | 20 | D | 174,185 | 22.2 | None |
| Control II^{c} | O-precursor | 20 | D | 154,160 +^{d} | 45.2 | None |
| FIFTEEN | O-3 | 10 | C | d | d | None |
| SIXTEEN | O-4 | 10 | A | d | d | None |
| SEVENTEEN^{e} | O-5 | 15 | C | 161 +^{d} | 0.85 | None |

*A = gummy; B = viscous liquid; C = fluid liquid; D = dispersion of solid particles in liquid.
^{a}For composition, see TABLE III.
^{b}zip Tₘ at about 229° C.
^{c}Controls I and II were made by mixing the precursor EC4 with zip.
^{d}Complex endotherm and respective J/g could not be determined with certainty.
^{e}prepared aseptically.

### EXAMPLE 7

### Characterization of Solubility of Ziprasidone from Typical Conjugates

For the solubility determination, all conjugate samples were placed in Eppendorf tubes with 1-ml aliquot of phosphate buffered saline (PBS), pH adjusted to 7.4. At selected time points (20 minutes, 1 hour, 6 hours, 24 hours, and 7 days), 200 □l of PBS exposed to each sample was withdrawn and replaced with fresh medium. The samples were continuously agitated for the duration of the study. To prepare HPLC samples, the 200-μl samples were filtered through 0.22-μm syringe, filter membrane, diluted as needed, and injected at appropriately adjusted volume to determine ziprasidone concentration in solution. Control I and Control II were used as controls because they were prepared using hydroxyl-ended polymers and no conjugation with the ziprasidone free base was expected as confirmed in the above characterization results (Table IV). The ionic conjugates evaluated for the solubility of ziprasidone in PBS are listed in Table V.

TABLE V
List of conjugates evaluated for solubility

| | | Drug Loading |
|---|---|---|
| Sample | Polymer | as Free Base |
| ONE | Malic acid initiated Lactide/Glycolide (B Series) | 25% |
| EIGHTEEN | | 10% |
| THIRTEEN | C-succinylated PEG 400/Trimethylene Carbonate/Caprolactone | 20% |
| FOURTEEN | (O Series) | 20% |
| FIFTEEN | C-succinylated PEG 600/Trimethylene Carbonate/Caprolactone | 10% |
| | (O Series) | |
| NINETEEN | C-succinylated PEG 600/Trimethylene Carbonate/Caprolactone | 10% |
| | (O Series) | |
| Control I | Not-succinylated PEG 400/Trimethylene Carbonate/Caprolactone | 20% |
| Control II | Not-succinylated PEG 400/Trimethylene Carbonate/Caprolactone | 20% |

Solubility results are summarized in Table VI. Conjugation appears to significantly increase aqueous solubility of ziprasidone as compared to both controls and free base and mesylate salt forms of ziprasidone. In general, the solubility of conjugates is higher than the solubility of controls, followed by the solubility of mesylate salt and free base of ziprasidone.

TABLE VI
Solubility of ziprasidone from liquid ionic conjugates compared to that of
the controls and solubility of ziprasidone free base and mesylate forms in PBS, pH 7.4.

| | | | Time Point/Sample Conc. [μg/ml] | | | | |
|---|---|---|---|---|---|---|---|
| Sample | Polymer | Drug Load | 20 min | 1 hour | 6 hours | 24 hours | 7 days |
| ONE | Malic acid initiated | 25% | 35.8 | 65.1 | 108.2 | 73.2 | 183.2 |
| EIGHTEEN | Lactide/Glycolide | 10% | 8.2 | 22.0 | 65.1 | 5.8 | 10.7 |
| THIRTEEN | C-succinylated PEG | 20% | 227.8 | 796.2 | 103.8 | 71.2 | 388.4 |
| | 400/Trimethylene | | | | | | |
| FOURTEEN | Carbonate/ | 20% | 1015.9 | 488.4 | 119.3 | 39.9 | 1651.8 |
| | Caprolactone | | | | | | |
| FIFTEEN | C-succinylated PEG | 10% | 33.2 | 55.9 | 793.7 | 912.3 | 1964.5 |
| | 600/Trimethylene | | | | | | |
| | Carbonate/ | | | | | | |
| | Caprolactone | | | | | | |
| NINETEEN | C-succinylated PEG | 10% | 22.4 | 12.1 | 52.6 | 150.9 | 1037.2 |
| | 600/Trimethylene | | | | | | |
| | Carbonate/ | | | | | | |
| | Caprolactone | | | | | | |
| Control I | Not-succinylated PEG | 20% | 11.1 | 14.7 | 35.7 | 11.2 | 6.5 |
| | 400/Trimethylene | | | | | | |
| | Carbonate/ | | | | | | |
| | Caprolactone | | | | | | |
| Control II | Not-succinylated PEG | 20% | 0.6 | 0.9 | 1.8 | 1.0 | 0.9 |
| | 400/Trimethylene | | | | | | |
| | Carbonate/ | | | | | | |
| | Caprolactone | | | | | | |
| Ziprasidone | Not applicable | 100% | 0.01 | — | 0.22 | 2.0 | 0.01 |
| Free Base | | | | | | | |
| Ziprasidone | Not applicable | 73% | 1.46 | — | 1.1 | — | 1.31 |
| Mesylate | | | | | | | |

What is claimed is:

1. A liquid conjugate comprising a bioactive agent and an absorbable liquid polymer, said bioactive agent and said absorbable liquid polymer being at least partly ionically bonded together to form said liquid conjugate.

2. The liquid conjugate of claim 1 wherein at least 50 percent of said bioactive agent and said absorbable liquid polymer is tonically bonded together.

3. The liquid conjugate as in claim 1 wherein the bioactive agent is a basic bioactive molecule and the polymer is a polycarbonate, polyester-carbonate, or polyester carrying one or more carboxyl group.

4. The liquid conjugate as in claim 1 wherein the liquid polymer component is a carboxyl-bearing segmented copolyester comprising a polyalkylene oxide segment.

5. The liquid conjugate as in claim 1 wherein the liquid polymer component is a carboxyl-bearing homopolymeric or copolymeric polyalkylene oxide.

6. The liquid conjugate composition as in claim 1 wherein said bioactive agent is ziprasidone and said liquid polymer is a carboxyl-bearing block/segmented copolymer comprising a polyalkylene carbonate and a polyalkylene oxide segment/block.

7. A pharmaceutical composition comprising the liquid conjugate of claim 1 and a pharmaceutically acceptable carrier.

8. The pharmaceutical composition of claim 7 wherein said pharmaceutically acceptable carrier is for controlled release or immediate release.

9. The pharmaceutical composition of claim 7 wherein said bioactive agent is an aryl-heterocyclic compound.

10. The pharmaceutical composition of claim 9 wherein said aryl-heterocyclic compound is ziprasidone.

11. The pharmaceutical composition of claim 7 wherein said liquid polymer is (i) a carboxyl-bearing polyester, copolyester, polyalkylene carbonate, copolyester carbonate or combinations thereof; or (ii) an amine-bearing polyester, copolyester, polyalkylene carbonate, polyether carbonate, polyether, copolyester-carbonate or combinations thereof.

12. The pharmaceutical composition of claim 7 wherein said bioactive agent is ziprasidone and said liquid polymer is a carboxyl-bearing block/segmented copolymer comprising a polyalkylene carbonate and a polyalkylene oxide segment/block.

13. A composition comprising a solid bioactive agent and one or more liquid polymers, wherein said bioactive agent and said liquid polymer or polymers comprise moieties, wherein said moieties of said bioactive agent interact in said composition with said moieties of said liquid polymer or polymers, wherein at least about 50 percent of said interaction is ionic bonding.
